(19) United States
(12) Patent Application Publication
(10) Pub. No.: US 2010/0159000 A1
(43) Pub. Date: Jun. 24, 2010

(54) Medicine for treating gastrointestinal disorder including fecal incontinence

(76) Inventor: James E. Lundeen, SR., Columbus, OH (US)

Correspondence Address: James E. Lundeen, Sr., M.D., 1430 S. High St., Columbus, OH, 43207, US

(21) Appl. No.: 12/660,552

(22) Filed: Mar. 1, 2010

Related U.S. Application Data
(63) Continuation-in-part of application No. 11/282,387, filed on Nov. 18, 2005, now abandoned.
Application No. 11/282,387 is a division of application No. 10/970,501, filed on Oct. 21, 2004, now abandoned.
(60) Provisional application No. 60/518,715, filed on Nov. 10, 2003.
(60) Provisional application No. 60/518,718, filed on Nov. 10, 2003.
(60) Provisional application No. 60/518,719, filed on Nov. 10, 2003.

(51) Int. Cl. A61K 31/55; A61K 9/48; A61K 9/28; A61P 1/00

(52) U.S. Cl. 424/456; 424/474; 514/217

(57) ABSTRACT

A method and a medicine for treating a human having a gastrointestinal disorder that includes fecal incontinence and/or urgency are provided. The method includes administering a dose of the medicine to the human. The medicine includes capsaicin (including one or more of: capsaicin, dihydrocapsaicin, nordihydrocapsaicin, homodihydrocapsaicin and homocapsaicin), a salt of imipramine and ducosate sodium.

### CROSS-REFERENCE TO RELATED APPLICATIONS

This application is a continuation-in-part and is filed under 37 CFR 1.53(b) and claims priority under 35 U.S.C. 120 to co-pending U.S. patent application Ser. No. 11/282,387, filed Nov. 18, 2005 which itself was a division of and claims priority under 35 U.S.C. 120 to U.S. application Ser. No. 10/970,501, filed 21 October 2004; which in turn claims priority under 35 U.S.C. 119(e) to U.S. Provisional Patent Applications Ser. Nos. 60/518,715 filed on November 10, 2003; 60/518,718 filed on Nov. 10, 2003; and 60/518,719 filed on Nov. 10, 2003, the disclosures of all of which are hereby incorporated by reference in their entirety.

### BACKGROUND

1. Field of Invention

The present invention generally relates to a method and a medicine for reducing or eliminating the undesirable affects of a gastrointestinal disorder. The present invention relates to a method for reducing or eliminating fecal incontinence and/or urgency associated with the gastrointestinal disorder. The present invention relates to a medicine for reducing or eliminating fecal incontinence and/or urgency associated with the gastrointestinal disorder.

2. Discussion of Related Art

Gastrointestinal disorders may manifest with one or a plurality of symptoms, including fecal incontinence and/or urgency. All further references to fecal incontinence are intended to mean fecal incontinence and/or urgency. Fecal incontinence may be either an acute functional disorder or a chronic functional disorder of the lower bowel. An acute attack may last only a few days or weeks, and is not recurring over a long period of time. In contrast, a chronic affliction may last or persist for a long time, or may reoccur regularly or irregularly over a long time.

The walls of the gastrointestinal (GI) tract have four layers: the mucosa, submucosa, muscularis externa, and serosa. The mucosa consists of an epithelium with basement membrane (called the lamina propria), loose connective tissue, blood vessels, and lymph tissues. The submucosa contains loose connective tissue, glands, nerves, and blood vessels. The nerve fibers of the submucosa form a network or plexus called the plexus of Meissner. The muscularis extema may include two bands of smooth muscle cells, the internal layer is composed of circular smooth muscle and the external layer is composed of longitudinal fibers. Interspersed between the muscle fibers is a nerve plexus called the plexus of Auerbach. The outermost layer of the digestive tube, the serosa, is composed of a membrane of squamous epithelium.

The gastrointestinal tract has a simplified “brain”, or nerve network, in the myenteric and submucosal plexuses, which has about 100 million neurons (the enteric nervous system). Efferents in the submucosal Meissner plexus regulate secretion by intestinal glands. The efferents in the myenteric Auerbach plexus control peristalsis, which is the rhythmic contraction of circular and longitudinal muscles. Visceral sensory afferent nerve endings are located throughout the submucosa and the Meissner plexus. Cranial nerves of the parasympathetic nervous system (e.g., the vagus) convey much of the sensory information from the gastrointestinal tract. However, sympathetic afferent nerves may transmit visceral sensations of pain to the spinal cord. Sensations, motility, digestion and secretion may be controlled by nerve elements of the gastrointestinal tract. The gastrointestinal tract submucosa contains sensory afferent nerve fibers that code for pressure, temperature and pain signals. Injury to the gastrointestinal tract may contribute to gastrointestinal disorders, such as fecal incontinence.

Treatment options for fecal incontinence may range from dietary modification and drug therapy to surgery. With respect to diet, the patient may avoid foods to which they possess a known sensitivity with respect to exacerbating the problem.

With reference to drugs, medicines and other treatments for fecal incontinence, few or none have demonstrated sufficient efficacy or sustainability to be of practical benefit to a majority of patients with chronic conditions over an extended period of time. Drugs and medicines directed to the gastrointestinal tract may include antacids, anti-spasmodic agents, anti-diarrheal drugs, anti-inflammatory drugs such as glucocorticosteroids and NSAIDS, histamine-R2-blocking agents, and antibiotics. Treatment may include surgery of the affected tissues, which may be an undesirable option.

Anti-spasmodic (anti-cholinergic) medication may be prescribed to decrease intestinal motility. U.S. Pat. Nos. 4,611,011, 4,701,457, and 4,745,131 disclose a series of amidinoureas which reduce intestinal motility. 1-Azabicyclo [2-2-2] octan-3-yl-2-aryl-3-azacyclo-2-hydroxy propionates and their quaternary salts, which possess antispasmodic activity, are disclosed in U.S. Pat. No. 4,843,074. Calcium channel antagonists exhibit muscle relaxing and antispasmodic activities. A series of substituted imidazolyl-alkyl-piperazine and diazepine derivatives, disclosed in U.S. Pat. No. 5,043,447, are calcium channel antagonists and may be useful as antispasmodics. U.S. Pat. No. 4,877,779 discloses 2-Aminomethylalkynylalkyl-1,3-dithiane derivatives with calcium-channel blocking activity and potentially similar uses. Some triazinone derivatives having spasmolytic activity are disclosed in U.S. Pat. No. 4,562,188.

In addition to antispasmodic agents, compounds with other activities have been disclosed which may relieve symptoms. Anti-diarrheal agents, such as loperamide, diphenoxylate, and codeine phosphate, have been used. Unfortunately, such agents are of little practical long-term benefit. Other anti-diarrheals include anti-cholinergics and smooth muscle relaxants, such as cimetropium bromide, pinaverium bromide, octilium bromide, trimebutine, and mebeverine.

Anti-spasmodics and anti-diarrheal preparations may be used to treat fecal incontinence. Even if effective, use of these preparations for long-term treatment may be precluded by problems such as development of tolerance, toxicity, or abuse potential.

Non-selective excitatory opioid receptor antagonists have been identified as central nervous system treatments that affect various gastrointestinal functions, and have been used to treat gastrointestinal disorders. Non-selective excitatory opioid receptor antagonists include, for example, tricyclic antidepressants, such as amitriptyline, imipramine, and doxepin. These opioid receptor antagonists may be effective for some gastrointestinal disorder symptoms due to the neuromodulatory and analgesic, properties of these compounds. However, the administration of the receptor antagonists has been precluded for long-term care for chronic conditions. Long term use of amitriptyline, imipramine or doxepin invariably leads to constipation and fecal impaction when administered alone.

While the manufacturer's recommended dosages of imipramine pamoate may be modified as necessary, the manufacturer's recommended dosages include: initial adult dosage for outpatients starting at 75 mg/day, which may be increased to 150 mg/day—the level at which the optimum response is usually obtained for anti-depression treatment. For anti-depression treatment, the manufacturer's recommended dosages for hospitalized patients start at an even higher dose of 100-150 mg/day—and the dosage can be raised as high as 300 mg/day. Elderly patients and children are stated as likely to respond to a dosage of about 25-50 mg/day per manufacturer's recommendations.

In contrast to the non-selective antagonists above, selective excitatory opioid receptor antagonists have been studied as treatments for gastrointestinal disorders. For example, U.S. Pat. No. 5,512,578 discloses co-administration of a selective excitatory opioid receptor antagonist with bimodally-acting opioid agonist, which may enhance potency of an analgesic, and may reduce tolerance and dependence liability. Such selective antagonists include, when administered at appropriately low doses, naloxone, naltrexone, etorphine, and dihydroetorphine. The selective excitatory opioid receptor antagonists attenuate excitatory, but not inhibitory, opioid receptor functions in nociceptive (pain) pathways of the peripheral and central nervous systems. As a result, symptoms associated with activation of excitatory opioid receptors, such as anti-analgesia, hyperalgesia, hyperexcitability, physical dependence and/or tolerance effects may be increased.

In spite of the many treatments, compositions and methods used to reduce or eliminate fecal incontinence, a suitable long-term efficacious treatment or preventative has not been identified. It may be desirable to have a medicinal composition or medicine having improved properties for the treatment of fecal incontinence and/or fecal urgency. It may be desirable to have improved or alternative methods of treatment for the treatment of fecal incontinence and/or fecal urgency.

### SUMMARY

The present invention relates to a method for treating a human having a gastrointestinal disorder. The disorder may include fecal incontinence and/or fecal urgency. The method includes administering a dose of the medicine to the human. The medicine includes the hydrochloride salt or pamoate salt of imipramine, capsaicin (including one or more of: capsaicin, dihydrocapsaicin, nordihydrocapsaicin, homodihydrocapsaicin and homocapsaicin) and the specific stool softener ducosate sodium.

An embodiment of the present invention relates to the medicine for treating a human having a gastrointestinal disorder that includes fecal incontinence. The medicine includes hydrochloride salt or pamoate salt of imipramine (which while classified as a tricyclic antidepressant, has specific properties as relates to this invention and a different tricyclic antidepressant cannot be effectively substituted in this invention, capsaicin (including one or more of: capsaicin, dihydrocapsaicin, nordihydrocapsaicin, homodihydrocapsaicin and homocapsaicin) and the specific stool softener ducosate sodium.

In one embodiment, the invention relates to a process that includes interacting with muscarinic receptors in the human to reduce or eliminate fecal incontinence and/or fecal urgency. In one embodiment, the process further includes both emulsifying and lubricating the fecal matter to both soften the stool and facilitate passage of the stool.

### BRIEF DESCRIPTION OF THE DRAWINGS

not applicable.

### DESCRIPTION OF PREFERRED EMBODIMENTS

The present invention relates to a method of treating a chronic disorder of the gastrointestinal (GI) tract with a medicinal composition. In one embodiment, the present invention relates to a method of treatment. In another embodiment, the present invention relates to a medicinal composition.

As used herein, a chronic condition refers to a condition that lasts for a substantial period or long time, and in some instances a chronic condition may not have an endpoint. Furthermore, chronic conditions may be continuous or recurring, and may reoccur regularly or irregularly. Gastrointestinal tract disorders include fecal urgency with or without fecal incontinence. Diarrhea is intended to broadly include both a medical practitioner's definition—an increased frequency of bowel movements, and a lay person's definition—liquid or fluid stool that causes difficulty of continence. A medicinal composition (“medicine”) is a substance administered in the treatment of a disorder; a remedial agent or a remedy; and a preventative. An efficacious amount is an amount greater than zero that has a desired or desirable effect.

Methods according to embodiments of the invention include the administration of a dose or a series of doses of the medicine to a patient suffering from or presenting symptoms associated with a gastrointestinal (GI) disorder. Dosage information is described below. The gastrointestinal disorder may include fecal incontinence and/or fecal urgency, and is the result of disease and/or injury to the lumbar spine and its elements (vertebrae, discs, ligaments, blood supply).

In each embodiment, the medicine includes hydrochloride salt or pamoate salt of imipramine, capsaicin (including one or more of: capsaicin, dihydrocapsaicin, nordihydrocapsaicin, homodihydrocapsaicin and homocapsaicin) and the specific stool softener ducosate sodium. The medicine according to embodiments of the present invention may be used to treat fecal incontinence. The specific stool softener ducosate sodium (that may individually otherwise increase stool frequency) is present regardless of frequent stools present at the time treatment is initiated. Adding the specific stool softener for this condition is not obvious to try, is counter-intuitive and the elements of this invention have a synergistic effect.

In each embodiment a salt of imipramine, capsaicin and docusate sodium are present. Imipramine hydrochloride is available for commercial sale as TOFRANIL from Mallinckrodt Inc. (St. Louis, Mo.). As used throughout, reference to dosage of imipramine generally will be to an equivalent amount of imipramine HCl. Also, unless specified, dosage values are in units of milligrams. For anti-depression treatment, manufacturer's supply TOFRANIL in 10, 25, 50 and 75 mg tablets or capsules. Imipramine pamoate is commercially available as TOFRANIL-PM from Mallinckrodt Inc. in capsules of 75 mg, 100 mg, 125 mg and 150 mg.

For an adult, the content of imipramine in a medicine according to an embodiment of the present invention will be 5 mg per dose. The content of capsaicin will be in measured in mg per dose and will vary with preparations varying from mild to moderate to higher doses. Capsaicin (including one or more of: capsaicin, dihydrocapsaicin, nordihydrocapsaicin, homodihydrocapsaicin and homocapsaicin) will be present in all preparations of this medicine. The ducosate sodium will be 100 mg per dose. The total daily dose will depend on the severity of symptoms and the number of doses, typically 1-3, required to ameliorate undesirable bowel symptoms of urgency and/or fecal incontinence.

The total daily dosage need not be taken at one time. Rather, the dosage may be taken as portions of the total daily dosage over the course of the day.

The specific stool softener, ducosate sodium, as used herein, is distinguished from laxatives and lubricants, neither of which is suitable for chronic usage.

By way of contrast, a stool softener acts to emulsify water and/or soap into fecal matter and thus soften the consistency. In each embodiment, the specific stool softener includes bis (2-ethylhexyl) sulfosuccinate sodium salt (“docusate sodium”), which is commercially available from Purdue Phama L.P. (Stamford, Conn.) as COLACE. The manufacturer recommended initial dosage level for COLACE is usually about 50-200 mg/day in divided doses.

The dosage amount of capsaicin varies according to intended potency of the embodiment. In one embodiment, the amount of capsaicin may be preselected based on weight, symptom severity, symptom type, symptom frequency, dietary considerations, dose regimen, administration method, environmental considerations, other or additional medications, and the like. In one embodiment, the amount may be selected based on individual responsiveness, dietary considerations, environmental considerations, side effects, aggravating conditions such as stress level, other or additional medications, and the like.

With reference to form of the medicine, it may be a coated pill, capsule, or gelcap.

The number of doses or portions taken per day may be adjusted to correspond to preselected factors. Such factors may include, for example, seasonal changes (e.g., dehydration, being more prevalent in summer months, may result in a temporary amelioration of fecal incontinence), aging, the natural course of the gastrointestinal disorder, stress inducing situations, and others that may affect the occurrence or severity of symptoms of the gastrointestinal disorder.

In patients who have had a colostomy or the stool bypasses the colon, the consistency of the stool may sometimes be undesirable. Sometimes a colostomy bag collects the stool using a stoma. Stool having an undesirable consistency may be difficult to handle and may irritate the skin adjacent the stoma because the fecal material has not been processed yet through the colon. Administration of the medicine according to the invention may improve the consistency of the fecal material. The small bowel may process the fecal material to a consistency similarly to a consistency of fecal material that had been processed by the colon. In one embodiment, in response to the administration of the medicine, the water content is reduced for fecal material entering a colostomy bag from a patient who has had a colostomy, and the fecal material may be emulsified with the water. The reduced water content, alone or in conjunction with the emulsification, may improve the consistency of the fecal material and the ease of handling of the fecal material.

The processes and embodiments described herein are examples of compositions, systems and methods having elements corresponding to the elements of the invention recited in the claims. This written description may enable those skilled in the art to make and use embodiments having alternative elements that likewise correspond to the elements of the invention recited in the claims. The intended scope of the invention thus includes other compositions, systems and methods that do not differ from the literal language of the claims, and further includes other compositions, systems and methods that are equivalent to, or have insubstantial differences from, the literal language of the claims.

What is claimed is:

1. A medicinal composition for treating a human having a gastrointestinal disorder comprising fecal incontinence, fecal urgency, or a combination thereof, the composition comprising: varying amounts of capsaicin, (including one or more of: capsaicin, dihydrocapsaicin, nordihydrocapsaicin, homodihydrocapsaicin and homocapsaicin) depending on strength of dosage, a salt of imipramine containing 5 mg of imipramine by weight, and 100 mg by weight of docusate sodium.

2. The composition as defined in claim 1, wherein the gastrointestinal disorder is a result of the human having disease and/or injury to the element(s) of the lumbar spine.

3. The composition as defined in claim 1, wherein the dose of capsaicin (including one or more of: capsaicin, dihydrocapsaicin, nordihydrocapsaicin, homodihydrocapsaicin and homocapsaicin) is present in an efficacious amount in a range of less than about 10,000 milligrams per total daily dose.

4. The composition as defined in claim 1, wherein medicine is configured as portions comprising fractional dosage amounts, the fractional dosage amounts being operable to effect variations in a total daily dosage amount of capsaicin, imipramine and ducosate sodium over a course of a daily treatment.

5. The composition as defined in claim 1, wherein at least a portion of the medicine is in the form of a pill, capsule, gelcap, an ingestible liquid admixture, transdermal patch, an oral or nasal inhalable powder or mist, an enema or suppository, a coated or chewable tablet, a chewable gum, an intravenous solution, or an intramuscular injectable liquid.

6. The composition as defined in claim 1, further comprising packaging, wherein the medicine is in the form of a plurality of co-packaged dosages of the medicine, and each dose comprises a portion of a daily dosage amount, wherein each dose is comprised of capsaicin (including one or more of: capsaicin, dihydrocapsaicin, nordihydrocapsaicin, homodihydrocapsaicin and homocapsaicin), an imipramine salt and ducosate sodium.

7. A treatment kit for a human having a gastrointestinal disorder comprising fecal incontinence, the kit comprising: a plurality of doses of a medicine, the medicine comprising: capsaicin (including one or more of: capsaicin, dihydrocapsaicin, nordihydrocapsaicin, homodihydrocapsaicin and homocapsaicin), salt of imipramine and ducosate sodium.

8. The kit as defined in claim 7, further comprising an instruction set comprising directions for administering the medicine, the instruction set comprising dosage amounts, dosing schedules, or both dosage amounts and dosing schedules.
